SEQUENCE LISTING (63 sequences)

SEQ ID NO: 1 - protein, 9 aa, synthetic construct
CHKKPSKSC

SEQ ID NO: 2 - protein, 9 aa, synthetic construct
CHRRPSRSC

SEQ ID NO: 3 - protein, 9 aa, synthetic construct
AHKKPSKSA

SEQ ID NO: 4 - protein, 14 aa, synthetic construct
MHGKTQATSGTIQS

SEQ ID NO: 5 - protein, 12 aa, synthetic construct
WALRRSIRRQSY

SEQ ID NO: 6 - protein, 12 aa, synthetic construct
WAGAKRLVLRRE

SEQ ID NO: 7 - protein, 12 aa, synthetic construct
LKAHLPPSRLPS

SEQ ID NO: 8 - protein, 8 aa, synthetic construct
VSGSSPDS

SEQ ID NO: 9 - protein, 9 aa, synthetic construct
RRTVKHHVN

SEQ ID NO: 10 - protein, 11 aa, synthetic construct
ACTARSPWICG

SEQ ID NO: 11 - protein, 12 aa, synthetic construct
MSPHPHPRHHHT

SEQ ID NO: 12 - protein, 19 aa, synthetic construct
SSKKSGSYSGSKGSKRRIL

SEQ ID NO: 13 - protein, 12 aa, synthetic construct
HPPMNASHPHMH

SEQ ID NO: 14 - protein, 6 aa, synthetic construct
RKLPDA

SEQ ID NO: 15 - protein, 9 aa, synthetic construct
CTYSRKHKC

SEQ ID NO: 16 - protein, 12 aa, synthetic construct
LRRSSEAHNSIV

SEQ ID NO: 17 - protein, 12 aa, synthetic construct
TSNAVHPTLRHL

SEQ ID NO: 18 - protein, 7 aa, synthetic construct
PTSTGQA

SEQ ID NO: 19 - protein, 7 aa, synthetic construct
TLTTLTN

SEQ ID NO: 20 - protein, 7 aa, synthetic construct
SSFPQPN

SEQ ID NO: 21 - protein, 11 aa, synthetic construct
CSQSVTSTKSC

SEQ ID NO: 22 - protein, 12 aa, synthetic construct
AYSSGAPPMPPF

SEQ ID NO: 23 - protein, 12 aa, synthetic construct
NPSSLFRYLPSD

SEQ ID NO: 24 - protein, 12 aa, synthetic construct
RPRENRGRERGL

SEQ ID NO: 25 - protein, 6 aa, synthetic construct
RKLPDA

SEQ ID NO: 26 - protein, 12 aa, synthetic construct
PPPWLPYMPPWS

SEQ ID NO: 27 - protein, 21 aa, synthetic construct
VKTQATSREEPPRLPSKHRPG

SEQ ID NO: 28 - protein, 12 aa, synthetic construct
EAHVMHKVAPRP

SEQ ID NO: 29 - protein, 7 aa, synthetic construct
EPLQLKM

SEQ ID NO: 30 - protein, 12 aa, synthetic construct
HSSYWYAFNNKT

SEQ ID NO: 31 - protein, 12 aa, synthetic construct
DYFSSPYYEQLF

SEQ ID NO: 32 - protein, 8 aa, synthetic construct
DSPHTELP

SEQ ID NO: 33 - protein, 20 aa, synthetic construct
LRNIRLRNIRLRNIRLRNIR

SEQ ID NO: 34 - protein, 40 aa, synthetic construct
IINNPITCMTNGAICWGPCPTAFRQIGNCGHFKVRCCKIR

SEQ ID NO: 35 - protein, 10 aa, synthetic construct
IINNPITCMT

SEQ ID NO: 36 - protein, 10 aa, synthetic construct
ITCMTNGAIC

SEQ ID NO: 37 - protein, 10 aa, synthetic construct
NGAICWGPCP

SEQ ID NO: 38 - protein, 10 aa, synthetic construct
WGPCPTAFRQ

SEQ ID NO: 39 - protein, 10 aa, synthetic construct
TAFRQIGNCG

SEQ ID NO: 40 - protein, 10 aa, synthetic construct
IGNCGHFKVR

SEQ ID NO: 41 - protein, 10 aa, synthetic construct
HFKVRCCKIR

SEQ ID NO: 42 - protein, 20 aa, synthetic construct
TAFRQIGNCGHFKVRCCKIR

SEQ ID NO: 43 - protein, 30 aa, synthetic construct
NGAICWGPCPTAFRQIGNCGHFKVRCCKIR

SEQ ID NO: 44 - protein, 19 aa, synthetic construct
IINNPITCMTNGAICWGPC

SEQ ID NO: 45 - protein, 30 aa, synthetic construct
IINNPITCMTNGAICWGPCPTAFRQIGNCG

SEQ ID NO: 46 - protein, 33 aa, synthetic construct
NGAICWGPCPTAFRQIGNCGHFKVRCCKIRDED

SEQ ID NO: 47 - protein, 5 aa, synthetic construct
ARLPR

SEQ ID NO: 48 - protein, 40 aa, synthetic construct
CIEQSFTTLFACQTAAEIWRAFGYTVKIMVDNGNCRLHVC

SEQ ID NO: 49 - protein, 35 aa, synthetic construct
IEEQAKTFLDKFNHEAEDLFYQSSLASWNYNTNIT

SEQ ID NO: 50 - protein, 12 aa, synthetic construct
WLVFFVIFYFFR

SEQ ID NO: 51 - protein, 12 aa, synthetic construct
WLVFFVIAYFAR

SEQ ID NO: 52 - protein, 16 aa, synthetic construct
WLVFFVIFYFFRRRKK

SEQ ID NO: 53 - protein, 16 aa, synthetic construct
RRKKWLVFFVIFYFFR

SEQ ID NO: 54 - protein, 10 aa, synthetic construct
RRKKIFYFFR

SEQ ID NO: 55 - protein, 10 aa, synthetic construct
WLVFFVRRKK

SEQ ID NO: 56 - protein, 10 aa, synthetic construct
FFVIFYRRKK

SEQ ID NO: 57 - protein, 15 aa, synthetic construct
QMRRKVELFTYMRFD

SEQ ID NO: 58 - protein, 7 aa, synthetic construct
NDFRSKT

SEQ ID NO: 59 - protein, 9 aa, synthetic construct
CNDFRSKTC

SEQ ID NO: 60 - protein, 20 aa, synthetic construct
GCKKYRRFRWKFKGKFWFWG

SEQ ID NO: 61 - protein, 19 aa, synthetic construct
GKKYRRFRWKFKGKWFWFG

SEQ ID NO: 62 - protein, 30 aa, synthetic construct
GFWFKGKWRFKKYRGGRYKKFRWKGKFWFG

SEQ ID NO: 63 - protein, 15 aa, synthetic construct
SSNKSTTGSGETTTA

(19) United States
(12) Patent Application Publication
(10) Pub. No.: US 2023/0295023 A1
(43) Pub. Date: Sep. 21, 2023

(54) Decontamination of Environmental Water Sources Using Living Engineered Biofilm Materials

(71) Applicant: ShanghaiTech University, Shanghai (CN)

(72) Inventors: Chao Zhong, Shanghai (CN); Jiahua Pu, Shanghai (CN); Yi Liu, Shanghai (CN)

(73) Assignee: ShanghaiTech University, Shanghai (CN)

(21) Appl. No.: 18/067,732

(22) Filed: Dec. 19, 2022

Related U.S. Application Data
(63) Continuation of application No. PCT/CN2021/108907, filed on Jul. 28, 2021.

(30) Foreign Application Priority Data
Jul. 28, 2020 (WO) PCT/CN2020/105078
Jul. 28, 2021 (WO) PCT/CN2021/108907

(51) Int. Cl. C02F 1/68; C02F 1/50

(52) U.S. Cl. CPC C02F 1/683; C02F 1/50; C02F 2101/10

(57) ABSTRACT

A living engineered biofilm material comprises microbial cells embedded in a protective extracellular matrix comprising a fusion protein of an amyloid domain and a contaminant binding domain operative to bind a contaminant of a water source, and thereby facilitate decontamination of the water source.

### REFERENCE TO A SEQUENCE LISTING

A Sequence Listing in XML format is incorporated by reference into the specification. The name of the XML file containing the Sequence Listing is STU19-001-2US.xml. The XML file is 55,735 bytes and was created on Dec. 17, 2022 and submitted electronically via EFS-Web on Dec. 19, 2022.

### INTRODUCTION

Waterborne disease outbreaks from viral pathogens occur each year worldwide¹, and the disinfection of viral pathogens is recognized as a critical but challenging process in water treatment^{2,3}. Conventional technologies to address this problem like chlorine and ozone treatment are chemically intensive and may produce dangerous disinfection by-products⁴, while use of UV light illumination and high-pressure filtration are energy intensive and can fail against UV-resistant viruses like adenovirus⁵. Further, some viruses are resistant to the chemicals used in water disinfections⁶, and the sizes of some viruses are too small to be filtered by conventional membranes^{7,8}. Thus, the development of a new generation of simple, efficient and environmentally friendly virus disinfection strategies that are complementary to existing technologies would be highly demanded. To this end, water treatment experts have suggested the exciting possibility of future technologies that might achieve exquisite molecular-level specificity for selective viral binding to materials functionalized with, for example, host receptor proteins of specific viruses².

Biofilms—consortia of microbial cells embedded in a protective extracellular matrix⁹-have been used in water treatment for a long time¹⁰. For example, naturally occurring biofilms are frequently harvested for the remediation of toxic compounds and heavy metals¹⁰. Inspired by these historical applications of biofilms in water treatment, we here propose and explore the concept of engineering biofilms as living materials for decontamination of water supplies contaminated with viral or bacterial pathogens, organic dyes, antibiotics, artificial sweeteners, pharmaceuticals, perfluorinated compounds, flame retardants, etc. based on the extracellular assembly of genetically engineered proteins in the biofilm matrix to enable specific interactions with and thus robust capture of targeted contaminants, such as pathogenic viruses. Ideally, such an approach would achieve highly efficient and selective disinfection of targeted viruses with very low energy inputs and minimal infrastructure requirements. Moreover, this living biofilm platform would harness the unique properties of living systems, including genetic programmability, self-regeneration, and evolutionary and environmental adaptability, attributes offering advantages over conventional water treatment technologies in terms of scalability for bio-manufacture and deployment, for example to prevent transmission of waterborne pathogens at geographically remote or otherwise inaccessible sites during epidemic outbreaks.

### SUMMARY OF THE INVENTION

The invention provides materials and methods for decontaminating water sources.

In an aspect the invention provides a living engineered biofilm material configured for decontamination of an environmental water source, wherein the biofilm material comprises microbial cells embedded in a protective extracellular matrix comprising a fusion protein expressed by the microbial cells, the fusion protein comprising an amyloid domain and a contaminant binding domain operative to bind a contaminant of the water source, and thereby facilitate decontamination of the water source.

In embodiments:
the contaminant is selected from a microbial (e.g. viral, bacterial, fungal or protozoan) pathogen, organic dye, antibiotic, artificial sweetener, pharmaceutical, perfluorinated compound, and flame retardant, and particularly a viral pathogen;
the contaminant is selected from a water-transmitted microbial pathogen that is a viral pathogen selected from Adenovirus, Astrovirus, Hepatitis A and E viruses, Rotavirns, Norovirus, Coxsackie viruses, Polioviruses, Polyomaviruses, Cytomegalovirus, Coronaviruses and Influenza viruses, or a bacterial pathogen selected from Aeromonas, Pseudomonas Salmonella, Aeromonas, Shigella, Vibrio spp., Enterobacter and Klebsiella, or a protozoan pathogen selected from Giardia lamblia and Schistosome;
the contaminant is a harmful chemical in water selected from arsenic, heavy metals, halogenated aromatics, nitrosoamines, nitrates and phosphates;
the microbial cells are Bacillus spp. (e.g. B.subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum)or Escherichia spp. (e.g. E. coli);
the amyloid domain is of TasA (B. subtilis), CsgA (E. coli), PSMs (S. aureus), RmbC (V. cholera), CsgA (Enterobacter cloacae), FapC (Pseudomonas spp.), CsgA (Salmonella spp.) or PAc (Streptococcus mutans);
the amyloid domain is a CsgA monomer;
the contaminant binding domain and contaminant are selected from:

| | |
|---|---|
| Contaminant binding domain peptide | Target contaminant |
| CHKKPSKSC (SEQ ID NO:1) | TiO2 binding |
| CHRRPSRSC (SEQ ID NO:2) | TiO2 binding |
| AHKKPSKSA (SEQ ID NO:3) | TiO2 binding |
| MHGKTQATSGTIQS (SEQ ID NO:4) | Gold binding |
| WALRRSIRRQSY (SEQ ID NO:5) | Gold binding |
| WAGAKRLVLRRE (SEQ ID NO:6) | Gold binding |
| LKAHLPPSRLPS (SEQ ID NO:7) | Gold binding |
| VSGSSPDS (SEQ ID NO:8) | Gold binding |
| RRTVKHHVN (SEQ ID NO:9) | Iron oxide |
| ACTARSPWICG (SEQ ID NO:10) | Lanthanide oxide and upconversion nanocrystals |
| MSPHPHPRHHHT (SEQ ID NO:11) | silica |
| SSKKSGSYSGSKGSKRRIL (SEQ ID NO:12) | silica |
| HPPMNASHPHMH (SEQ ID NO:13) | silica |
| RKLPDA (SEQ ID NO:14) | silica |
| CTYSRKHKC (SEQ ID NO:15) | Cadmium sulphide |
| LRRSSEAHNSIV (SEQ ID NO:16) | Zinc sulphide |
| TSNAVHPTLRHL (SEQ ID NO:17) | Palladium binding |
| PTSTGQA (SEQ ID NO:18) | Platinum binding |
| TLTTLTN (SEQ ID NO:19) | Platinum binding |
| SSFPQPN (SEQ ID NO:20) | Platinum binding |
| CSQSVTSTKSC (SEQ ID NO:21) | Platinum binding |
| AYSSGAPPMPPF (SEQ ID NO:22) | silver |
| NPSSLFRYLPSD (SEQ ID NO:23) | silver |
| RPRENRGRERGL (SEQ ID NO:24) | Titanium |
| RKLPDA (SEQ ID NO:25) | Titanium |
| PPPWLPYMPPWS (SEQ ID NO:26) | Quartz |
| VKTQATSREEPPRLPSKHRPG (SEQ ID NO:27) | Zeolites |
| EAHVMHKVAPRP (SEQ ID NO:28) | Zinc oxide |
| EPLQLKM (SEQ ID NO:29) | Graphene |
| HSSYWYAFNNKT (SEQ ID NO:30) | Single-walled carbon nanotubes |
| DYFSSPYYEQLF (SEQ ID NO:31) | Single-walled carbon nanotubes |
| DSPHTELP (SEQ ID NO:32) | Single-walled carbon nanotubes; |

the contaminant is a viral pathogen, and the contaminant binding domain and contaminant are selected from:

| | |
|---|---|
| Contaminant binding domain peptide | Contaminant |
| LRNIRLRNIRLRNIRLRNIR (SEQ ID NO:33) | hepatitis B virus |
| IINNPITCMTNGAICWGPCPTAFRQIGNCGHFKVRCCKIR (SEQ ID NO:34) IINNPITCMT (SEQ ID NO:35) ITCMTNGAIC (SEQ ID NO:36) NGAICWGPCP (SEQ ID NO:37) WGPCPTAFRQ (SEQ ID NO:38) TAFRQIGNCG (SEQ ID NO:39) IGNCGHFKVR (SEQ ID NO:40) HFKVRCCKIR (SEQ ID NO:41) TAFRQIGNCGHFKVRCCKIR (SEQ ID NO:42) NGAICWGPCPTAFRQIGNCGHFKVRCCKIR (SEQ ID NO:43) IINNPITCMTNGAICWGPC (SEQ ID NO:44) IINNPITCMTNGAICWGPCPTAFRQIGNCG (SEQ ID NO:45) NGAICWGPCPTAFRQIGNCGHFKVRCCKIRDED (SEQ ID NO:46) | influenza A virus H1N1, H3N2, H5N1, H7N7, H7N9, SARS-CoV and MERS-CoV |
| ARLPR (SEQ ID NO:47) (C5) | H1N1 |
| CIEQSFTTLFACQTAAEIWRAFGYTVKIMVDNGNCRLHVC (SEQ ID NO:48) (C40) | H1N1 |
| IEEQAKTFLDKFNHEAEDLFYQSSLASWNYNTNIT (SEQ ID NO:49) | Sars-Cov-2 |
| WLVFFVIFYFFR (SEQ ID NO:50) WLVFFVIAYFAR (SEQ ID NO:51) WLVFFVIFYFFRRRKK (SEQ ID NO:52) RRKKWLVFFVIFYFFR (SEQ ID NO:53) RRKKIFYFFR (SEQ ID NO:54) WLVFFVRRKK (SEQ ID NO:55) FFVIFYRRKK (SEQ ID NO:56) | H1N1 |
| QMRRKVELFTYMRFD (SEQ ID NO:57) | Enterovirus 71 |
| NDFRSKT (SEQ ID NO:58) | H9N2 |
| CNDFRSKTC (SEQ ID NO:59) | H9N3 |
| GCKKYRRFRWKFKGKFWFWG (SEQ ID NO:60) | H7N7 |
| GKKYRRFRWKFKGKWFWFG (SEQ ID NO:61) | H3N2 |
| GFWFKGKWRFKKYRGGRYKKFRWKGKFWFG (SEQ ID NO:63) | H1N1 |
| SSNKSTTGSGETTTA (SEQ ID NO:63) | H1N1; and/or |

the contaminant binding domain is an influenza virus hemagglutinin binding peptide selected from: ARLPR (SEQ ID NO:47) (C5) and IEQSFTTLFACQTAAEIWRAFGYTVKIMVDNGNCRLHVC (SEQ ID NO:48) (C40).

In an aspect the invention provides a water decontamination system comprising a subject living engineered biofilm material and a water source comprising the contaminant.

In an aspect the invention provides a water decontamination system comprising an industrial filler material colonized by a subject living engineered biofilm material.

In an aspect the invention provides a method of using a subject living engineered biofilm material, comprising the step of contacting a water source comprising the contaminant with the biofilm material under conditions wherein the fusion protein binds the contaminant.

The invention includes all combinations of recited particular embodiments as if each combination had been laboriously recited.

### BRIEF DESCRIPTION OF THE FIGURES

FIG. 1. Integrating engineered functional biofilms with industrial filler materials for virus elimination from river water; schematic for polypropylene industrial filler material colonized by our engineered CsgA-C5 biofilms and used to eliminate viruses from river water.

FIG. 2. qPCR analysis of field samples after virus-spiked river water samples were passed over the immobilized biofilms. Results show means ± s.e.m.

FIG. 3. Immunofluorescence images of the biofilms-coated polypropylene industrial filler material after passage of the field water samples, stained against hemagglutinin. The insert image refers to the bare filler materials as a control test sample.

FIG. 4. SEM images of the virus particles bound to the CsgA-C5 biofilms (zoomed-in images are shown at the right). E. coli cells, amyloid fibers, and virus particles are indicated with arrows.

### DESCRIPTION OF PARTICULAR EMBODIMENTS OF THE INVENTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Our living engineered biofilms can be applied in disinfection of viral pathogens in water treatment.

Our engineered biofilms can also be designed to decontaminate other pollutes in water source, including antibiotics, organic dyes, heavy metals, water-borne viruses (hepatitis E, enterovirus, adenovirus, etc), bacteria (Staphylococcus aureus, Vibrio cholera, etc) and protozoans (Giardia lamblia, Schistosome). Detailed information is listed in Table 1.

Our engineered biofilms can be coated onto industrial fillers (https://en.wikipedia.org/wiki/Filler (materials)) easily, making our living materials complementary to existing industrial water treatment infrastructure.

Given that biofilm disinfection materials can be grown as needed in situ, they may be easier to distribute to remote areas (where various target-pathogen-functionalized biofilms could be stored as culture sample libraries), especially in difficult-to-access areas during epidemic outbreaks.. That is, rather than requiring the transport of dangerous chemicals, energy-intensive filtration equipment, strong generators, and trained personal to properly and safely implement and manage pathogen-disinfection water treatment processes, local inhabitants of such areas could for example grow living engineered biofilms in their own buckets and other water vessels.

### Example: Virus Disinfection From Environmental Water Sources

Using Living Engineered Biofilm Materials. In this example we disclose a simple, efficient, and environmentally friendly approach to achieve highly selective disinfection of viruses with living engineered biofilm materials. As as initial proof-of-concept, we designed fusion monomer proteins that comprise an amyloid CsgA domain to facilitate nanofiber self-assembly and a peptide (C5) known to specifically bind the influenza-virus-surface protein hemagglutinin (HA). We revealed that the fusion CsgA-C5 proteins, endogenously expressed and secreted by Escherichia coli cells, could self-assemble into biofilm matrix and eventually capture virus particles directly from aqueous solutions, disinfecting samples to a level below the limit-of-detection for a qPCR-based detection assay. By exploiting the surface-adherence properties of biofilms, we further showed that polypropylene filler materials colonized by the CsgA-C5 biofilms could be utilized to disinfect river water samples with influenza titers as high as 1×10⁷ PFU/L. Our example demonstrates that engineered biofilms can be harvested for the disinfection of pathogens from environmental water samples and highlighted the unique biology-only properties of living substances for material applications.

Our rational design for engineering E. coli biofilms for disinfection of virus in water was based on curli-specific gene (csg) products, CsgA proteins, a major component of E. coli biofilms¹¹. CsgA protein monomers are secreted from bacterial cells and can self-assemble into amyloid nanofibers¹². Notably, genetically modified E. coli biofilms have recently found a wide range of interesting applications in catalysis, biosensor and bioremediation as engineered living materials¹³⁻¹⁶. As an initial proof-of-concept for viral binding in this study, we choose the influenza virus (H1N1) as a model, and engineered fusion monomers that combined CsgA with a known influenza-virus-binding peptide—here denoted as C5. The C5 peptide, previously identified by phage display¹⁷, was rationally fused with the CsgA protein to form CsgA-C5 fusion monomer. CsgA-C5 proteins can be secreted out of the bacteria cells and self-assemble into the amyloid fibers comprising the extracellular matrix of engineered biofilms.

We initially used computational approaches to assess the reactivity of CsgA-C5 fusion monomers. Although previous work has shown that the C5 influenza-virus-binding peptide has a high affinity to hemagglutinin, we needed to confirm that C5 could still interact with hemagglutinin after being fused with the CsgA protein. To such ends, we first used MODELLER^{18,19} to build the homology models of CsgA-C5 and Glide²⁰ to get the complex of the monomer CsgA-C5 and hemagglutinin (PDB ID: 1HGG). Molecular dynamics simulations of the interaction between a CsgA-C5 fusion monomer and hemagglutinin by GROMACS²¹ indicated that these two proteins interact strongly: the bound complex structure was stable even after 800 ns. The interactions among the key residues include hydrogen bonding interactions (between R136 (CsgA-C5) and S136 (HA), between R136 (CsgA-C5) and E190 (HA)) and hydrophobic interactions (between L134 (CsgA-C5) and K156 (HA), among P135 (CsgA-C5),W153(HA) and L194 (HA), between R136 (CsgA-C5) and L226 (HA)). The binding energy was calculated using the MM/GBSA (Molecular Mechanics/Generalized Born Surface Area) method²², and the ΔG_{bind} value was about -62±22 kcal/mol, which is similar to the binding energy between biotin analogous and avidin²³.

We used E. coli to recombinantly express CsgA monomers and CsgA-C5 monomers, and following cell lysis, these proteins were purified following standard protocols¹⁹ and migrated as single bands at 14.1 kDa and 14.6 kDa, respectively, under SDS-polyacrylamide gel electrophoresis (SDS-PAGE) and western blotting. We then conducted QCM (quartz crystal microbalance) experiments wherein fresh eluted CsgA and fusion CsgA-C5 monomers were exposed to silicon substrates that were coated with hemagglutinin beforehand. Compared with CsgA control monomers, CsgA-C5 monomers showed substantially enhanced absorption: the mass of CsgA-C5 monomers absorbed on the HA-coated substrate was about 75% greater than the mass of the absorbed CsgA monomers. This result indicates that the C5 peptide is essential for the interaction between CsgA and hemagglutinin, and confirms that CsgA-C5 fusion monomers retain the hemagglutinin-binding activity of the C5 peptide.

We next investigated whether the presence of the C5 peptide might affect the overall structure of CsgA amyloid cores. We again initially built molecular dynamics models: one representing the monomeric and one representing the fibrillar states of the CsgA-C5 structures. Simulations of the monomeric proteins (1 µs) and the fibrillar states (1 µs) indicated that the core amyloid structure comprising the CsgA-C5 fusion monomers does not substantially diverge from that of a typical CsgA amyloid structure. The models also suggested that CsgA-C5 monomers should assemble into stable amyloid structures dominated by the CsgA domains, with the C5 peptides displayed external to the amyloid core. Collectively, these results thus validate the rationality of our design-the influenza virus-binding sites are fully exposed, which should allow binding of influenza hemagglutinin with the C5 peptide of the fibrillar amyloids.

To experimentally validate the results from our simulations, we next tested if the CsgA-C5 fusion monomer proteins could assemble into fibers. ThT (thioflavin T) and Congo red assays showed that CsgA-C5 and CsgA proteins exhibited amyloid features. Further, both the CsgA-C5 and CsgA monomers were able to self-assemble into long and stable fibers, as confirmed by TEM (transmission electron microscopy) and AFM (atomic force microscopy). In addition, X-ray fiber diffraction data revealed the typical cross-beta diffraction patterns characteristic of amyloid structures²⁴ for both the CsgA-C5 and CsgA amyloid nanofibers.

We next used immunofluorescence and enzyme-linked immunosorbent assay (ELISA) to test if the self-assembled amyloid nanofibers retained their capacity for binding hemagglutinin: both analytical methods showed that the affinity of CsgA-C5 fibers for hemagglutinin was increased by 10 fold compared to CsgA fibers. We also investigated if CsgA-C5 fibers can bind with intact virus particles. TEM images demonstrated that apparently all of the CsgA-C5 protein fibers could specifically bind with influenza virus particles; this was in sharp contrast to the CsgA-His controls, which absorbed very few virus particles. Immunofluorescence microscopy images also showed a similar result: the glass substrate coated with CsgA-C5 fibers was covered with viruses, whereas very few viruses were absorbed on the CsgA-coated glass. Quantification using ELISA and immunofluorescence intensity analysis showed that the affinity of CsgA-C5 nanofibers for hemagglutinin was about 4 fold greater than that of the CsgA fibers.

Having established at the protein level that our CsgA-C5 amyloid fibers form strong interactions with influenza virus particles, we next explored the use of engineered E. coli biofilms with the CsgA-C5 fibers to capture viruses directly from aqueous solutions. The virus particles were added to the culture media and then co-cultured with the engineered E. coli cells. Prior to induction of biofilm formation, the virus particles were freely distributed around bacteria cells. After induction for 72 hours, we found that the CsgA-C5 fibers of the adhered E. coli biofilms bound with many influenza virus particles. In contrast, very few virus particles were captured by control CsgA fibers in biofilms.

We next explored the influenza-virus-binding capacity of the engineered biofilms by exposing them to a series of influenza virus titers (ranging from 2.9 ×10⁴ to 2.9 ×10⁵ PFU/mL). We collected the supernatants from the samples, and AFM analysis showed that there were many virus particles in the control supernatants (from CsgA biofilm samples) but hardly any in the supernatant from the CsgA-C5 biofilm samples, even at very high viral titers. Further, ELISA detected a clear concentration-dependent increase in viral signal for the control samples that were not exposed to biofilms (3 days at 29° C.), while only a very low signal was detected for the supernatants from the CsgA-C5 biofilm samples, with a slight increase evident for only the highest titer sample. We also analyzed these samples with the sensitive qPCR assay and noted the same trend: only the highest viral titer biofilm-incubated sample had a signal above the detection limit for the commercial Venzyme Cham QTM SYBR® Color qPCR Master Mix kit that we applied for this analysis.

Notably, both the ELISA and qPCR analyses revealed a small increase in the viral signal for the highest viral titer sample, suggesting possible saturation of the viral-particle-binding capacity of the C5 peptides present in these biofilms. Although it is difficult to precisely control the spatial distribution of C5 monomers in these living biofilms, it should in theory be possible to combine different bacterial seeding rates with similar viral concentration series to more precisely ascertain the saturation levels of these materials. Importantly considering the potential water-pathogen-disinfectant applications, we also tested whether mammalian cells could still become infected after virus-infected water was treated with the engineered CsgA-C5 biofilms. Specifically, we exposed the highly influenza-susceptible MDCK (Madin-Darby canine kidney) cells to control or post-biofilm incubation supernatant from the 7×10⁴ PFU/mL viral titer samples, and immunofluorescence analysis with an antibody against influenza virus nucleoprotein indicated that no cells became infected with the post-biofilm incubation supernatant. In sharp contrast, many of the cells exposed to the control supernatant had strong signals indicating virus infection. A similar result was also confirmed by hemagglutination inhibition assay in which the sediment of chicken red blood cells to the well bottom, a sign indicating the absence of viral HA proteins in the solution, was found for both of the biofilm-treated and virus-free negative control solutions. Taken together, these results revealed that our engineered biofilms could bind and thus efficiently eliminate influenza viral particles from aqueous solutions to an extent that apparently precludes infection of highly susceptible mammalian cells.

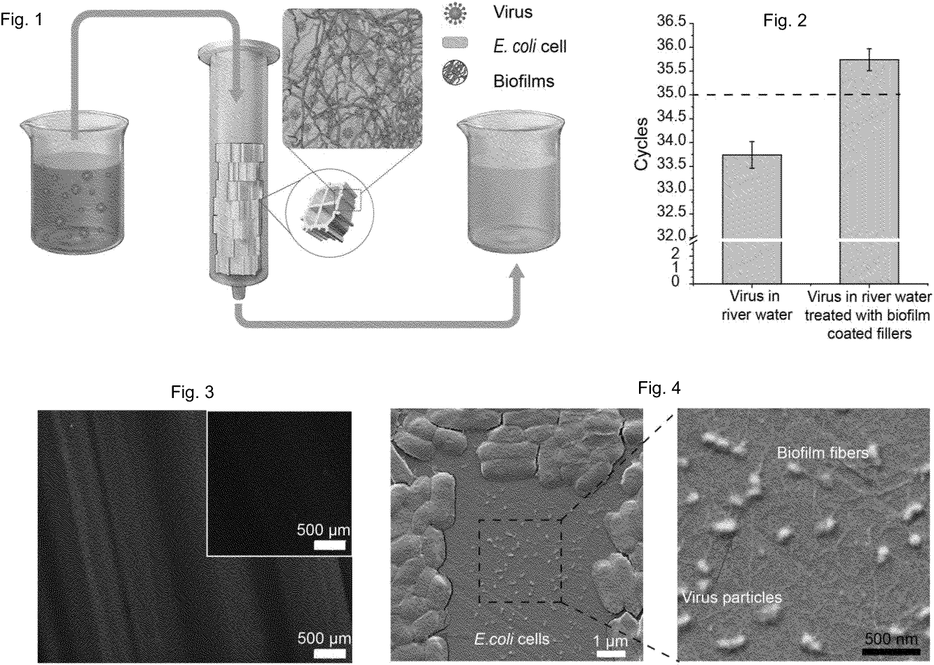

By exploiting the fact that E. coli biofilms can inherently adhere to diverse substances and complex structures^{25,26}, we next grew CsgA-C5 biofilms on the polypropylene surfaces of intricate gear-like industrial filler material and tested their capacity to capture virus particles from river water (FIG. 1). Congo red staining confirmed that the CsgA-C5 biofilms could successfully grow on the surfaces. We spiked river water samples with an influenza virus titer of 1×10⁷ PFU/L, a level much higher than reported human virus titers in river water (which range from 10²-10⁵ PFU/L)^{27,28}. We then passed the water samples over the filter materials, and qPCR analysis showed that the virus could be easily detected in the unfiltered control samples (virus-spiked river water) but was undetectable in the filtrate sample (FIG. 2). Further, both immunofluorescence (FIG. 3) and SEM (FIG. 4) images demonstrated that the virus particles from the river water samples had been attached to the nanofibers of the filter-immobilized CsgA-C5 biofilms.

We here used biofilms programmed with a virus-protein-binding peptide to endow engineered biofilms the ability to efficiently capture viruses from river water. Compared with conventional water treatment methods, our strategy is green, low costs, and low energy inputs. Essentially, our engineered CsgA-C5 biofilms achieved strong and specific disinfection of the target virus from water samples to a level that precluded infection of cells known to be highly susceptible to influenza infection. Extending the concept to showcase the biology-specific functional properties of a genuinely living material, we used our functional biofilms to grow on and colonize polypropylene inserts in a way that also robustly disinfected viruses from river water samples. Our initial proof-of-concept demonstrations targeted the influenza virus as a model, but clearly any virus-binding peptide or protein-based moiety (e.g., host receptor proteins, antibodies) should be suitable for fusion with CsgA proteins to enable similar biofilm-mediated disinfection for other viruses.

Given that bacterial biofilms can be genetically modified and considering the modularity of fusion amyloid monomers, it should be relatively straightforward to deploy other CsgA fusion proteins which combine the amyloid nanofiber self-assembly and biofilm-forming capacities of CsgA domains with additional functional domains that selectively bind to (and thus sequester) other viruses and perhaps even other classes of waterborne pathogens (e.g., binding to surface or other exposed proteins of bacteria like Vibrio cholera²⁹ and protozoans like Giardia lamblia³⁰). It should also be possible to further engineer the material performance of the biofilms themselves by fusing the CsgA-pathogen binding monomers to other protein domains that can alter biofilm mechanical properties tailored for specific applications. Looking beyond E. coli and in light of our previously reported work demonstrating that the FDA generally-regarded-as-safe bacterium Bacillus subtilis and its TasA amyloid proteins can be engineered and generally functionalized as a biofilm living material platform³¹, we anticipate that this generally pathogen-binding biofilm concept could even find applications in other application domains, for example, applying engineered biofilms in the gut to capture and digest toxic gut pathogens or viruses.

Our living materials are complementary to existing conventional technologies used for water disinfection, and an important point of contrast of our living materials versus conventional technologies like hazardous chemical treatment or ultra-filtration relates to technological deployment. Given that biofilm disinfection materials can be grown as needed in situ, they may be easier to distribute to remote areas (where various target-pathogen-functionalized biofilms could be stored as culture sample libraries), especially in difficult-to-access areas during epidemic outbreaks. Rather than requiring the transport of dangerous chemicals, energy-intensive filtration equipment, strong generators, and trained personal to properly and safely implement and manage pathogen-disinfection water treatment processes, local inhabitants of such areas could for example grow living engineered biofilms in their own buckets and other water vessels.

References

| | |
|---|---|
| 1 | Gibson, K. E. Viral pathogens in water: occurrence, public health impact, and available control strategies. Curr Opin Virol 4, 50-57, doi:10.1016/j.coviro.2013.12.005 (2014). |
| 2 | Mark A. Shannon, P. W. B., Menachem Elimelech, John G. Georgiadis, Benito J. Marinas & Anne M. Mayes. Science and Technology for Water Purification in the Coming Decade. nature 452 (2008). |
| 3 | Gall, A. M., Marinas, B. J., Lu, Y. & Shisler, J. L. Waterborne Viruses: A Barrier to Safe Drinking Water. PLoS Pathog 11, e1004867, doi:10.1371/journal.ppat.1004867 (2015). |
| 4 | Stuart W. Krasner, H. S. W., Susan D. Richardson, Salvador J. Pastor, Russell Chinn, Michael J. Sclimenti, Gretchen D. Onstad, and Alfred D. Thruston, JR. Occurrence of a new generation of disinfection byproducts. Environmental Science & Technology 40, 7175-7185 (2006). |
| 5 | Gerba, C. P., Gramos, D. M. & Nwachuku, N. Comparative inactivation of enteroviruses and adenovirus 2 by UV light. Appl Environ Microbiol 68, 5167-5169, doi:10.1128/aem.68.10.5167-5169.2002 (2002). |
| 6 | Sherri A. Tyrrell, S. R. R. a. W. D. W. Inactivation of bacterial and viral indicators in secondary sewage effluents, using chlorine and ozone. water reaserch 29 (1995). |
| 7 | Nwachcuku, N. & Gerba, C. P. Emerging waterborne pathogens: can we kill them all? Curr Opin Biotechnol 15, 175-180, doi:10.1016/j.copbio.2004.04.010 (2004). |
| 8 | Madaeni, S. S. The application of membrane technology for water disinfection. Water reaserch 33 (1999). |
| 9 | Flemming, H. C. et al. Biofilms: an emergent form of bacterial life. Nat Rev Microbiol 14, 563-575, doi: 10.1038/nrmicro.2016.94 (2016). |
| 10 | Nilanjana Das, L. V. G. B., Jaseetha Abdul Salam, Evy Alice Abigail M. Application of Biofilms on Remediation of Pollutants An Overview. Journal of Microbiology and Biotechnology Research 2 (2012). |
| 11 | Barnhart, M. M. & Chapman, M. R. Curli biogenesis and function. Annu Rev Microbiol 60, 131-147, doi: 10.1146/annurev.micro.60.080805.142106 (2006). |
| 12 | Chapman, M. R. et al. Role of Escherichia coli curli operons in directing amyloid fiber formation. Science 295, 851-855, doi:10.1126/science.1067484 (2002). |
| 13 | Xinyu Wang, J. P., Yi Liu, Fang Ba, Mengkui Cui, Ke Li, Yu Xie, Yan Nie, Qixi Mi, Tao Li, Lingli Liu, Manzhou Zhu, Chao Zhong. Immobilization of functional nano-objects in living engineered bacterial biofilms for catalytic applications. National Science Review (2019). |
| 14 | Cao, Y. et al. Programmable assembly of pressure sensors using pattern-forming bacteria. Nat Biotechnol 35, 1087-1093, doi:10.1038/nbt.3978 (2017). |
| 15 | Cao, B., Hu, Y. & Mukherjee, M. in ABSTRACTS OF PAPERS OF THE AMERICAN CHEMICAL SOCIETY. (AMER CHEMICAL SOC 1155 16TH ST, NW, WASHINGTON, DC 20036 USA). |
| 16 | Gilbert, C. & Ellis, T. Biological Engineered Living Materials: Growing Functional Materials with Genetically Programmable Properties. ACS Synth Biol 8, 1-15, doi:10.1021/acssynbio.8b00423 (2019). |
| 17 | Matsubara, T. et al. Sialic acid-mimic peptides as hemagglutinin inhibitors for antiinfluenza therapy. J Med Chem 53, 4441-4449, doi:10.1021/jm1002183 (2010). |
| 18 | Sali, A. Comparative protein modelling by satisfaction of spatial restraints. Molecular Medicine Today (1995). |
| 19 | Zhong, C. et al. Strong underwater adhesives made by self-assembling multi-protein nanofibres. Nat Nanotechnol 9, 858-866, doi:10.1038/nnano.2014.199 (2014). |
| 20 | Woody Sherman, T. D., Matthew P. Jacobson, Richard A. Friesner, and Ramy Farid. Novel Procedure for Modeling Ligand Receptor Induced Fit Effects. Journal of Medicinal Chemistry 49 (2006). |
| 21 | Abraham, M. J. et al. GROMACS: High performance molecular simulations through multi-level parallelism from laptops to supercomputers. SoftwareX 1-2, 19-25, doi:10.1016/j.softx.2015.06.001 (2015). |
| 22 | Huang, N., Kalyanaraman, C., Bernacki, K. & Jacobson, M. P. Molecular mechanics methods for predicting protein-ligand binding. Physical Chemistry Chemical Physics 8, 5166-5177 (2006). |
| 23 | Genheden, S. & Ryde, U. Comparison of the Efficiency of the LIE and MM/GBSA Methods to Calculate Ligand-Binding Energies. J Chem Theory Comput 7, 3768-3778, doi:10.1021/ct200163c (2011). |
| 24 | Greenwald, J. & Riek, R. Biology of amyloid: structure, function, and regulation. Structure 18, 1244-1260, doi:10.1016/j.str.2010.08.009 (2010). |
| 25 | Wang, X. et al. Programming Cells for Dynamic Assembly of Inorganic Nano-Objects with Spatiotemporal Control. Adv Mater 30, e1705968, doi:10.1002/adma.201705968 (2018). |
| 26 | Elizabeth P. DeBenedictis, J. L., Sinan Keten. Adhesion mechanisms of curli subunit CsgA to abiotic surfaces. Science advances 2 (2016). |
| 27 | Haramoto, E., Kitajima, M., Katayama, H. & Ohgaki, S. Real-time PCR detection of adenoviruses, polyomaviruses, and torque teno viruses in river water in Japan. Water Res 44, 1747-1752, doi:10.1016/j.watres.2009.11.043 (2010). |
| 28 | Hamza, I. A. et al. Detection of human viruses in rivers of a densly-populated area in Germany using a virus adsorption elution method optimized for PCR analyses. Water Res 43, 2657-2668, doi:10.1016/j.watres.2009.03.020 (2009). |
| 29 | Wang, J. et al. On the antigenic determinants of the lipopolysaccharides of Vibrio cholerae O: 1, serotypes Ogawa and Inaba. Journal of Biological Chemistry 273, 2777-2783 (1998). |
| 30 | Smith, P. D., Gillin, F. D., Brown, W. R. & Nash, T. E. IgG antibody to Giardia lamblia detected by enzyme-linked immunosorbent assay. Gastroenterology 80, 1476-1480 (1981). |
| 31 | Huang, J. et al. Programmable and printable Bacillus subtilis biofilms as engineered living materials. Nat Chem Biol 15, 34-41, doi:10.1038/s41589-018-0169-2 (2019). |

1. A living engineered biofilm material configured for decontamination of an environmental water source, wherein the biofilm material comprises microbial cells embedded in a protective extracellular matrix comprising a fusion protein expressed by the microbial cells, the fusion protein comprising an amyloid domain and a contaminant binding domain operative to bind a contaminant of the water source, and thereby facilitate decontamination of the water source.

2. The material of claim 1, wherein the contaminant is selected from a microbial (e.g. viral, bacterial, fungal or protozoan) pathogen, organic dye, antibiotic, artificial sweetener, pharmaceutical, perfluorinated compound, and flame retardant.

3. The material of claim 1, wherein the contaminant is a water-transmitted microbial pathogen that is a viral pathogen selected from Adenovirus, Astrovirus, Hepatitis A and E viruses, Rotavirns, Norovirus, Coxsackie viruses, Polioviruses, Polyomaviruses, Cytomegalovirus, Coronaviruses and Influenza viruses, or a bacterial pathogen selected from Aeromonas, Pseudomonas Salmonella, Aeromonas, Shigella, Vibrio spp., Enterobacter and Klebsiella, or a protozoan pathogen selected from Giardia lamblia and Schistosome.

4. The material of claim 1, wherein the contaminant is a harmful chemical in water selected from arsenic, heavy metals, halogenated aromatics, nitrosoamines, nitrates and phosphates.

5. The material of claim 1, wherein the contaminant binding domain and contaminant are selected from:

| | |
|---|---|
| Contaminant binding domain | Target contaminant |
| CHKKPSKSC (SEQ ID NO:1) | TiO2 binding |
| CHRRPSRSC (SEQ ID NO:2) | TiO2 binding |
| AHKKPSKSA (SEQ ID NO:3) | TiO2 binding |
| MHGKTQATSGTIQS (SEQ ID NO:4) | Gold binding |
| WALRRSIRRQSY (SEQ ID NO:5) | Gold binding |
| WAGAKRLVLRRE (SEQ ID NO:6) | Gold binding |
| LKAHLPPSRLPS (SEQ ID NO:7) | Gold binding |
| VSGSSPDS (SEQ ID NO:8) | Gold binding |
| RRTVKHHVN (SEQ ID NO:9) | Iron oxide |
| ACTARSPWICG (SEQ ID NO:10) | Lanthanide oxide and upconversion nanocrystals |
| MSPHPHPRHHHT (SEQ ID NO:11) | silica |
| SSKKSGSYSGSKGSKRRIL (SEQ ID NO:12) | silica |
| HPPMNASHPHMH (SEQ ID NO:13) | silica |
| RKLPDA (SEQ ID NO:14) | silica |
| CTYSRKHKC (SEQ ID NO:15) | Cadmium sulphide |
| LRRSSEAHNSIV (SEQ ID NO:16) | Zinc sulphide |
| TSNAVHPTLRHL (SEQ ID NO:17) | Palladium binding |
| PTSTGQA (SEQ ID NO:18) | Platinum binding |
| TLTTLTN (SEQ ID NO:19) | Platinum binding |
| SSFPQPN (SEQ ID NO:20) | Platinum binding |
| CSQSVTSTKSC (SEQ ID NO:21) | Platinum binding |
| AYSSGAPPMPPF (SEQ ID NO:22) | silver |
| NPSSLFRYLPSD (SEQ ID NO:23) | silver |
| RPRENRGRERGL (SEQ ID NO:24) | Titanium |
| RKLPDA (SEQ ID NO:25) | Titanium |
| PPPWLPYMPPWS (SEQ ID NO:26) | Quartz |
| VKTQATSREEPPRLPSKHRPG (SEQ ID NO:27) | Zeolites |
| EAHVMHKVAPRP (SEQ ID NO:28) | Zinc oxide |
| EPLQLKM (SEQ ID NO:29) | Graphene |
| HSSYWYAFNNKT (SEQ ID NO:30) | Single-walled carbon nanotubes |
| DYFSSPYYEQLF (SEQ ID NO:31) | Single-walled carbon nanotubes |
| DSPHTELP (SEQ ID NO:32) | Single-walled carbon nanotubes. |

6. The material of claim 1, wherein the contaminant is a viral pathogen and the contaminant binding domain and contaminant are selected from:

| | |
|---|---|
| Contaminant binding domain peptide | Contaminant |
| LRNIRLRNIRLRNIRLRNIR (SEQ ID NO:33) | hepatitis B virus |
| IINNPITCMTNGAICWGPCPTAFRQIGNCGHFKVRCCKIR (SEQ ID NO:34) | influenza A virus H1N1, |
| IINNPITCMT (SEQ ID NO:35) | H3N2, |
| ITCMTNGAIC (SEQ ID NO:36) | H5N1, |
| NGAICWGPCP (SEQ ID NO:37) | H7N7, |
| WGPCPTAFRQ (SEQ ID NO:38) | H7N9, |
| TAFRQIGNCG (SEQ ID NO:39) | SARS-CoV and MERS-CoV |
| IGNCGHFKVR (SEQ ID NO:40) | |
| HFKVRCCKIR (SEQ ID NO:41) | |
| TAFRQIGNCGHFKVRCCKIR (SEQ ID NO:42) | |
| NGAICWGPCPTAFRQIGNCGHFKVRCCKIR (SEQ ID NO:43) | |
| IINNPITCMTNGAICWGPC (SEQ ID NO:44) | |
| IINNPITCMTNGAICWGPCPTAFRQIGNCG (SEQ ID NO:45) | |
| NGAICWGPCPTAFRQIGNCGHFKVRCCKIRDED (SEQ ID NO:46) | |
| ARLPR (SEQ ID NO:47) (C5) | H1N1 |
| CIEQSFTTLFACQTAAEIWRAFGYTVKIMVDNGNCRLHVC (SEQ ID NO:48) (C40) | H1N1 |
| IEEQAKTFLDKFNHEAEDLFYQSSLASWNYNTNIT (SEQ ID NO:49) | Sars-Cov-2 |
| WLVFFVIFYFFR (SEQ ID NO:50) | H1N1 |
| WLVFFVIAYFAR (SEQ ID NO:51) | |
| WLVFFVIFYFFRRRKK (SEQ ID NO:52) | |
| RRKKWLVFFVIFYFFR (SEQ ID NO:53) | |
| RRKKIFYFFR (SEQ ID NO:54) | |
| WLVFFVRRKK (SEQ ID NO:55) | |
| FFVIFYRRKK (SEQ ID NO:56) | |
| QMRRKVELFTYMRFD (SEQ ID NO:57) | Enterovirus 71 |
| NDFRSKT (SEQ ID NO:58) | H9N2 |
| CNDFRSKTC (SEQ ID NO:59) | H9N3 |
| GCKKYRRFRWKFKGKFWFWG (SEQ ID NO:60) | H7N7 |
| GKKYRRFRWKFKGKWFWFG (SEQ ID NO:61) | H3N2 |
| GFWFKGKWRFKKYRGGRYKKFRWKGKFWFG (SEQ ID NO:63) | H1N1 |
| SSNKSTTGSGETTTA (SEQ ID NO:63) | H1N1. |

7. The material of claim 1, wherein the contaminant binding domain is an influenza virus hemagglutinin binding peptide selected from: ARLPR (SEQ ID NO:47) (C5) and CIEQSFTTLFACQTAAEIWRAFGYTVKIMVDNGNCRLHVC (SEQ ID NO:48) (C40).

8. The material of claim 1, wherein the amyloid domain is of TasA (B. subtilis), CsgA (E. coli), PSMs (S. aureus), RmbC (V. cholera), CsgA (Enterobacter cloacae), FapC (Pseudomonas spp.), CsgA (Salmonella spp.) or PAc (Streptococcus mutans).

9. The material of claim 1, wherein the amyloid domain is a CsgA monomer.

10. The material of claim 1, wherein the microbial cells are Bacillus spp. (e.g. B.subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

11. The material of claim 8, wherein the microbial cells are Bacillus spp. (e.g. B.subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

12. The material of claim 9, wherein the microbial cells are Bacillus spp. (e.g. B.subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

13. The material of claim 1 configured in a water decontamination system comprising the living engineered biofilm material, the system further comprising a water source comprising the contaminant.

14. The material of claim 1 configured in a water decontamination system comprising an industrial filler material colonized by the living engineered biofilm material.

15. The material of claim 10 configured in a water decontamination system comprising an industrial filler material colonized by the living engineered biofilm material.

16. The material of claim 11 configured in a water decontamination system comprising an industrial filler material colonized by the living engineered biofilm material.

17. The material of claim 12 configured in a water decontamination system comprising an industrial filler material colonized by the living engineered biofilm material.

18. The material of claim 1 configured in a water decontamination system comprising an industrial filler material colonized by the living engineered biofilm material, the system further comprising a water source comprising the contaminant.

19. A water decontamination system comprising an industrial filler material colonized by the living engineered biofilm material of claim 1.

20. A method of using the living engineered biofilm material of claim 1, comprising the step of contacting a water source comprising the contaminant with the biofilm material under conditions wherein the fusion protein binds the contaminant.
